(19) United States
(12) Patent Application Publication
(10) Pub. No.: US 2009/0281301 A1
(43) Pub. Date: Nov. 12, 2009

(54) Manufacturing Process of 2' ,2' - Difluoronucleoside and Intermediate

(75) Inventors: Moon-Sung Kim, Gyeonggi-do (KR); Yong-Jik Kim, Gyeonggi-do (KR); Jun-Ho Choi, Gyeonggi-do (KR); Hong-Gyu Lim, Seoul (KR); Dae-Won Cha, Gyeonggi-do (KR)

Correspondence Address: THE NATH LAW GROUP, 112 South West Street, Alexandria, VA, 22314, US

(73) Assignee: DONG-A-PHARM.CO LTD, Seoul (KR)

(21) Appl. No.: 12/086,337

(22) PCT Filed: Dec. 11, 2006
(86) PCT No.: PCT/KR2006/005372

§ 371 Date: Jun. 11, 2008

(30) Foreign Application Priority Data
Dec. 14, 2005 (KR) 10-2005-0123229
Dec. 11, 2006 (KR) 10-2006-0125230

(51) Int. Cl. C07H 19/06; C07D 307/26

(52) U.S. Cl. 536/28.53; 549/313

(57) ABSTRACT

The present invention relates to more improved process for preparing 2′-deoxy-2′,2′-difluoronucleoside and its intermediate. The present invention provide a process for preparing an erythro enantiomer in greater than 98% purity, comprising forming a lactone ring by hydrolyzing ethyl (3RS)-2,2-difluoro-3-hydroxy-3-(2,2-dimethyloxolan-4-yl)propionate is hydrolyzed in the presence of hydrolysis reagents selected from acetic acid or chloroacetic acid, water and a mixture of organic solvents selected from the group comprising acetonilrile, dioxane, tetrahydrofuran or toluene, introducing a substituted benzoyl protecting group at the 3-position and 5-position, and recrys- tallizing said erythro enantiomer. Further, the present invention provides a process for selectively preparing, in greater than 99% purity, a beta-anomer 2′-deoxy-2′,2′-difluoronucleoside at the 3′-position and 5′-position that are protected by a substituted benzoyl in a 2:3 alpha/beta anomeric ratio.

### TECHNICAL FIELD

The present invention relates to a novel process for preparing 2′,2′-difluoronucleoside and its intermediate of the following formula 1 that exhibits superior antitumor activity.

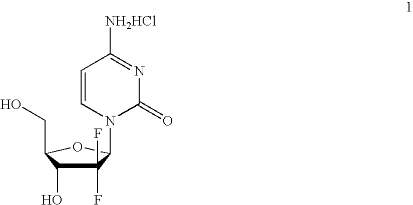

### BACKGROUND ART

2′-deoxy-2′,2′-difluoronucleoside of the above formula 1 is disclosed in European Patent Application No. 184,365 that describes the use of the same compounds as oncolytic agents. Currently, the compound has been shown to be effective for the treatment of non-small cell lung cancer, pancreatic cancer, bladder cancer and metastatic breast cancer.

U.S. Pat. Nos. 4,526,988 and 4,808,614 disclose the preparation of 2′-deoxy-2′,2′-difluoronucleoside, as shown in the following reaction scheme 1.

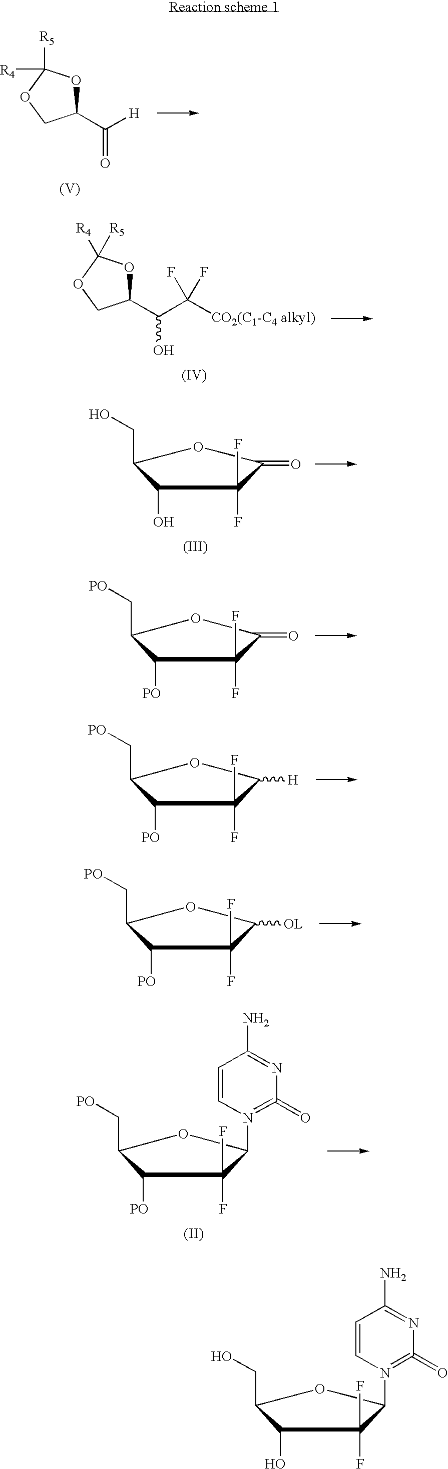

Wherein, R₄ and R₅ are independently C₁—C₃ alkyl; P is a hydroxy protecting group; and L is a leaving group.

A carbohydrate which has the stereochemistry of ribose is preferred since it provides 2′-deoxy-2′,2′-difluoronucleoside which exhibit superior biological activity. The intermediate lactone compound (III) of the prior art may be obtained in a mixture of erythro and threo stereoisomers.

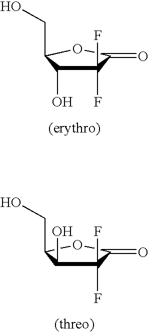

The prior art discloses that the erythro enantiomer is preferred since it provides a carbohydrate which has the stereochemistry of naturally occurring ribose.

The prior art also discloses the preparation of the above described erythro enantiomer by first forming an alkyl

2,2-difluoro-3-hydroxy-3-(2,2-dialkyldioxolan-4-yl)propionate, consisting of 3-R- and
3-S-hydroxy enantiomers of the formula IV compound, in a ratio of about 3 parts
3-R-enantiomer to about 1 part 3-S-enantiomer.

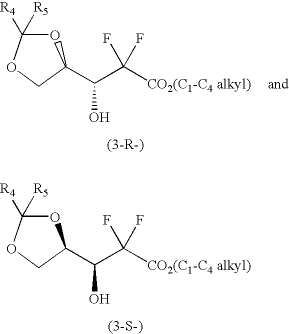

The prior art also describes the 3-R-hydroxy enantiomer has the proper stereo-chemistry to provide the desired erythro diastereomer and that the 3-R- and 3-S-enantiomers can be separated by expensive, laborious column chromatography procedures. Once the 3-R-hydroxy enantiomer is isolated it is next hydrolyzed under acidic conditions to form an unprotected lactone; namely,

2-deoxy-2,2-difluoro-D-erythro-pentofuranos-l-ulose, which has the formula III.

The beta-anomer precursor of the formula IT is preferred since it provides 2′-deoxy-2′,2′-difluoronucleoside which possess superior biological activity. The prior art specifically illustrates the use of tertiary-butyldimethylsilyl as a protecting group.

When this protecting group is used in the synthesis of

2′-deoxy-2′,2′-difluoronucleoside, the product is composed of about a 4:1 alpha/beta anomeric ratio. This product must be purified by expensive, laborious column chromatography procedures to isolate the desired beta-anomer in low yield.

More improved process of the prior art is described in the Korean Patent Examined Publication No. 1997-2659. The patent provides a process for obtaining 2′-deoxy-2′,2′-difluoronucleoside having the erythro- and beta-stereochemistry which eliminates the need for expensive column chromatography purification, as shown in the following reaction scheme 2.

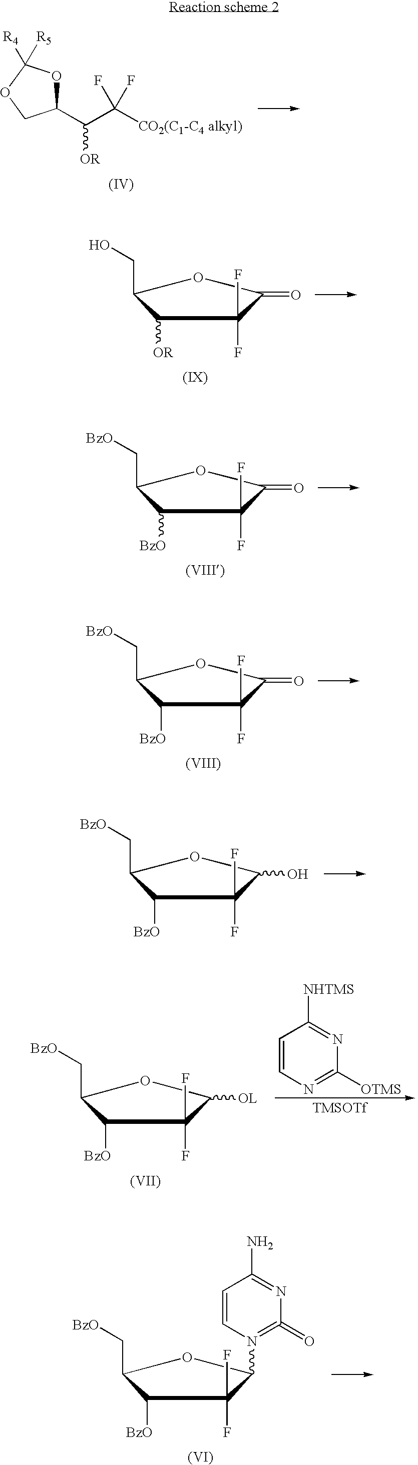

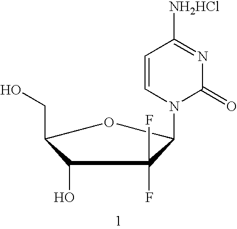

Wherein, R is H or

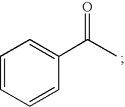

### Bz is

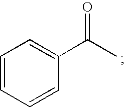

F₄ and R₅ are independently C₁-C₃ alkyl.

The process requires strong acids as hydrolysis reagents in hydrolyzing a compound of the formula IV to obtain a compound of the formula IX, a mixture of erythro and threo lactones.

Nevertheless, the above process for manufacturing the compound of the formula IX was carried out in a manner that it is heated under reflux at 78° C. for 8 hours. As a result, the compound of the formula IX was extremely instable under such stress conditions with poor yield.

Although a mixture of erythro and threo lactones is recrystallized and separated to prepare a pure compound of the formula VIII, the purity of erythro lactone is confined to 95%. Thus the Korean patent causes the formation of undesirable reaction products, making it difficult to obtain a pure 2′-deoxy-2′,2′-difluoronucleoside.

In addition, the Korean Patent provides a process for preparing 2′-deoxy-2′,2′-difluoronucIeoside, comprising reacting a compound of the formula VII with an appropriate base B-H, forming a compound of the formula VI, and removing the benzoyl protecting group by reacting with a base.

However, the Korean patent provides a process for selectively isolating 2′-deoxy-2′,2′-difluoronucleoside from a 1:1 alpha/beta anomeric ratio, with unnecessary alpha-anomer containing more than 50%. The process also requires an expensive reagent such as trimethylsilyl trifluoroacetate, when the compound of the formula VII is reacted with base B-H.

The Korean patent provides a process for selectively isolating 2′-deoxy-2′,2′-difluoronucleoside having the beta-stereochemistry in approximately 99% purity by utilizing a hydrochloride of the 1:1 alpha/beta anomeric mixture as starting material, dissolving the mixture in hot water, adding acetone and collecting the precipitated solids several times. However, the purification process requires several recrystallization processes to ensure better purity, which is less economical due to a poor yield following repeated recrystallization processes.

The Korean Patent Registered Publication No. 424990 provides a process for separating and purifying 2′-deoxy-2′,2′-difluoronucleoside.

The process employs alpha-anomer carbohydrate or alpha-anomer enriched carbohydrate in glycosylation process of a base and carbohydrate.

The Korean Patent Registered Publication No 302087 provides a process tor preparing the alpha-anomer carbohydrate, comprising preparing a carbohydrate with alpha- and beta-anomers at a low temperature and separating the alpha-anomer via recrystallization process.

However, the process is not economically feasible due to a low yield of 35.5˜68% with no reproducibility.

Although a mixture of beta-anomer enriched nucleosides is made available from glycosylation reaction between the alpha-anomer enriched carbohydrate and base, about 4:6 alpha-beta anomeric ratio is observed via high pressure liquid chromatography analysis.

In this context, any glycosylation reaction appears to be unnecessary due to a poor yield (68%) of the alpha-anomer carbohydrate, when isolated. In carrying out such reaction, a toxic anisole is employed as a reaction solvent having a boiling point of 154° C. Since anisole cannot be easily eliminated after reaction, the purity of 2′-deoxy-2′,2′-difluoronucleoside will be affected by the remaining solvent.

### DISCLOSURE OF INVENTION

### Technical Problem

An object of the present invention is to provide a process for preparing 2′-deoxy-2′,2′-difluoronucleoside of the following formula 1, using a pure intermediate which has the stereochemistry of naturally occurring ribose.

Another object of the present invention is to provide a process for obtaining, in greater than 99.9% purity, 2′-deoxy-2′,2′-difluoronucleoside of the following formula I by removal of protecting groups.

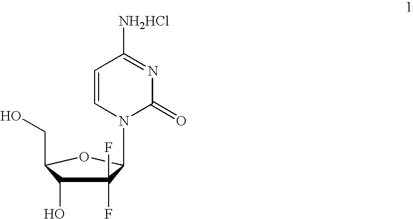

### Technical Solution

The present invention provides not only a process for preparing a novel intermediate by introducing a substituted benzoyl group as a novel protecting group, but also a purification process for obtaining, in greater than 99% purity, the beta-anomer via N-glycosylation reaction.

Further, the present invention provides a process for selectively obtaining, in greater than 99.9% purity, 2′-deoxy-2′,2′-difluoronucleoside hydrochloride of the formula 1 by removal of protecting groups.

The present invention is described in more detail as set forth hereunder.

The manufacturing method of the present invention is briefly illustrated, as shown in the following reaction scheme 3:

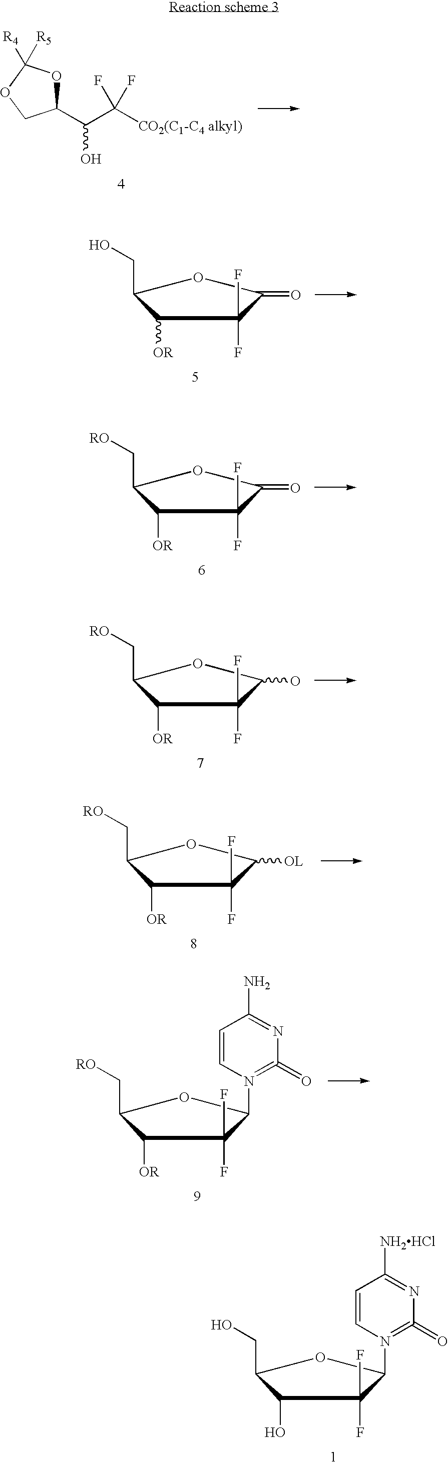

Wherein, R is

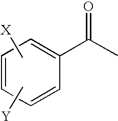

or H; X is F, Cl, Br, I, and NO₂, respectively; Y is H, F, Cl, Br, I and NO₂, respectively; and it is preferred that X and Y are a benzoyl derivative substituted at the 3-position or 5-position. Further, L is methanesulfonyl, p-toluenesulfonyl; R₄ and R₅ are independently C₁—C₃ alkyl.

The present invention provides a process for synthesizing a new intermediate (compound of the formula 6) by introducing a novel protecting group, substituted benzoyl group, from the compound of the formula 4.

The lactone compound of the formula 6 may be obtained from the compound of the formula 4 under mild conditions using weak acids or relatively strong acids as hydrolysis reagents in place of strong acids.

The compound of the formula 5, which is synthesized using the strong acids as hydrolysis reagents, is decomposed in the reaction due to instability in the strong acids that may result in poor yield.

According to the present invention, the term “weak acids or relatively strong acids” as hydrolysis reagents refers to acetic acid or chloroacetic acid.

The hydrolysis reagents of the present invention may include acetic acid, water and a mixture of organic solvents in a given ratio.

The acetic acid mixed with water comprises 10˜95% acetic acid. The organic solvent may be selected from the group comprising acetonitrile, dioxane, tetrahydrofuran and toluene. Acetic acid, organic solvent and water may be mixed in the weight ratio of 10˜95:0˜70:5˜90.

To prepare 2′-deoxy-2′,2′-difluoronucleoside in high purity, an object of the present invention is to provide a process for synthesizing a pure intermediate of the formula 6which has the stereochemistry of naturally occurring ribose should be obtained.

Accordingly, the present invention provides a process for obtaining a compound of the following formula 6′ having an enantiomer mixture of erythro and threo lactones via introduction of a substituted benzoyl protecting group.

In particular, once the unprotected hydroxy groups of the above lactone ring at the 3-position or 5-position are protected with substituted benzoyl groups such as halogen or nitro (electron withdrawing groups) in place of a benzoyl group, the erythro enantiomer can be rapidly isolated in the reaction. Thus a compound of the formula 6 may be easily prepared with a substituted benzoyl group of the present invention.

When the compound of the following formula 6′ having an enantiomer mixture of erythro and threo lactones, which is protected by a substituted benzoyl group, is purified through a recrystallization process, the erythro lactone of the formula 6 in high yield may be selectively isolated compared with the convention compound protected by benzoyl group.

The present invention may include ethyl acetate and hexane or heptane as recrystallization solvents. The present invention provides a process for obtaining, in greater than 98% purity, the desired erythro lactone protected by a substituted benzoyl group, as shown below.

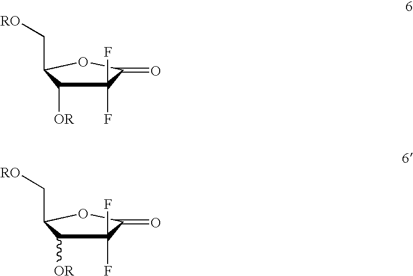

Wherein, R is

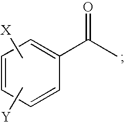

X is F, Cl, Br, I, and NO₂, respectively; Y is H, F, Cl, Br, I and NO₂, respectively; and it is preferred that X and Y are benzoyl derivatives substituted at the 3-position or 5-position. Further. L is methanesulfonyl or p-toluenesulfonyl.

As demonstrated in reaction scheme 3, the compound of the formula 6 is converted to a compound of the formula 8 by processes well known to those skilled in the art (Synthesis 1992, 565); hence, the preferred leaving group is methanesulfonate.

Further, the present invention provides a glycosylation process, wherein the protected carbohydrate of the formula 9 is reacted with silylated base in the absence of an expensive reagent such as tiimethylsilyl or trifluoroacetate, as well as a process for carrying out the reaction using a carbohydrate in a 1:1 alpha/beta anomeric ratio in the absence of a high boiling point solvent such as anisole.

According to the present invention, oxygen atoms are preferably enolized with the silyl protecting groups in order to increase the base's aromaticity and thereby allow more ready attack of the base by the carbohydrate in the glycosylation reaction.

To ensure better selectivity in the glycosylation reaction, the present invention provides a process for synthesizing the compound of the formula 9 in about 2:3 alpha/beta anomeric ratio, comprising adding a carbohydrate to base silylated by silylation reagents without using additional solvents or removing silylation reagents. The examples of silylation reagents include hexamethyldisilazane (HMDS) and bistrimethylsilylacetamide (BSA). The reaction is carried out at the temperature in the range of 60˜160° C., preferably in the range of 120˜140° C. The reaction is actually completed for about 4˜72 hours.

Further, the present invention provides a process for obtaining, in greater than 99% purity, a beta-anomer 2′-deoxy-2′,2′-difluoronucleoside of the formula 9 from 2′-deoxy-2′,2′-difluorocytidine-3′,5′-D-(substituted)-benzoate in a 2:3 alpha/beta anomeric ratio. The recrystallization process may be carried out using recrystallization solvents such as methanol, ethanol, 2-propanol, ethyl acetate, chlorform and methylene chloride; hence, it is more preferred to employ ethyl acetate.

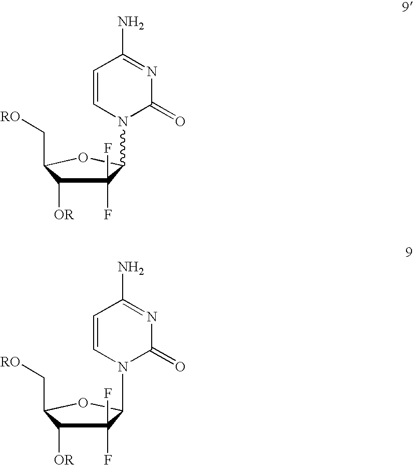

Wherein, R is

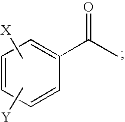

X is F, Cl, Br, I, and NO₂, respectively; Y is H, F, Cl, Br, I and NO₂, respectively; and it is preferred that X and Y are benzoyl derivatives substituted at the 3-position or 5-position. Further, L is methanesulfonyl, p-toluenesulfonyl.

Therefore, the present invention provides a novel process for selectively preparing, in greater than 99.9% purity, a beta-anomer 2′-deoxy-2′,2′-difluorocytidine hydrochloride, comprising removing the protecting groups of pure 2′-deoxy-2′,2′-difluorocytidine-3′,5′-D-(substituted)-benzoate using ammonia by processes well known to those skilled in the art to obtain an beta-anomer 2′-deoxy-2′,2′-difluorocytidine, dissolving the beta-anomer

2′-deoxy-2′,2′-difluorocytidine in ethanol by heating and adding an equimolar strong acid to give a beta-anomer 2′-deoxy-2′,2′-difluorocytidine hydrochloride.

### ADVANTAGEOUS EFFECTS

The present invention provides not only a process for preparing a novel intermediate by introducing a substituted benzoyl group as a novel protecting group, but also a purification process for obtaining, in greater than 99% purity, the beta-anomer via N-glycosylation reaction.

Further, the present invention provides a process for selectively obtaining, in greater than 99.9% purity, 2′-deoxy-2′,2′-difluoronucleoside hydrochloride of the formula 1 by removal of protecting groups.

### BEST MODE FOR CARRYING OUT THE INVENTION

This invention will now be described by reference to the following examples and experimental examples which are merely illustrative and which are not to be construed as a limitation of the scope of this invention.

### Example 1

Preparation of 2-deoxy-2,2-difluoro-1-oxoribose

To ethyl (3R,S)-2,2-difluoro-3-hydroxy-3-(2,2-dimethyloxolan-4-yl)propionate (30 g, 0.118 mole) were added acetonitrile (165 mL), acetic acid (67.6 mL) and water (11.7 mL) for mixing. The mixture was heated under reflux for 4 hours with stirring. With the addition of toluene (165 mL), the resulting solution was evaporated under reduced pressure. With the addition of acetonitrile (165 mL), the concentrate was distilled with toluene (300 mL) and evaporated under reduced pressure. Ethyl acetate (200 mL) was added to the concentrate for dilution and then, an active charcoal (3 g) was added to the diluted solution and stirred for 10 minutes. The resulting solution was dried over anhydrous sodium sulfate and filtered with diatomite. The residue was evaporated under reduced pressure to give a desired 2-deoxy-2,2-difluoro-1-oxoribose (20 g, 100%).

1H-NMR (DMSO d₆)δ:3.6˜3.8 (m, 2H), 4.2˜4.3 (m, 1H), 4.3˜4.5 (m, 1H)

### Example 2

Preparation of 2-deoxy-2,2-difluoro-D-erythro-3,5-bis-(3-fluorobenzoyloxy)-pentofuranos-1-ulose

A mixture of 4-dimethylaminopyridine (29 g), pyridine (28 g) and 3-fluorobenzoyl chloride (2.5 g) was added to 2-deoxy-2,2-difluoro-1-oxoribose (20 g, 0.119 mole) in ethyl acetate (200 mL). The mixture was stirred for 60° C. overnight. With completion of the reaction, the reaction mixture was washed with a weak solution of hydrochloric acid and saturated saline solution. The organic layer was dried over anhydrous sodium sulfate, filtered and evaporated under reduced pressure. The concentrate was diluted with ethyl acetate (23 mL) and with the addition of hexane (68 mL), cooled to 0° C. The crystals, so formed, were filtered, washed with a mixing solution of ethyl acetate:hexane (1:3; v:v) and dried to give a desired 2-deoxy-2,2-difluoro-D-erythro-3,5-bis-(3-fluorobenzoyloxy)-pentofuranos-1-ulose (26.7 g, 46%).

1H-NMR (CDCl₃)δ:4.69˜4.73 (dd, J=1.2 Hz, 2H), 4.96 (q, 1H), 5.72 (m, 1H), 7.24˜7.49 (m, 4H), 7.66˜8.86 (m, 4H)

### Example 3

Preparation of 2-deoxy-2,2-difluoro-3,5-bis-(3-fluorobenzoyloxy)-D-ribofuranose

To 2-deoxy-2,2-difluoro-D-erythro-3,5-bis-(3-fluorobenzoyloxy)-pentofuranos-1-ulose (24 g, 0.058 mole) were added tetrahydrofuran (240 mL) and lithium tri-tert-butoxyaluminohydride (22.2 g, 0.087 mole). The solution was stirred at room temperature for 30 minutes. With completion of the reaction, the solution was diluted with ethyl acetate (960 mL) and washed with a weak solution of hydrochloric acid, saturated sodium carbonate solution, water and saline solution successively. The mixture was dried over anhydrous sodium sulfate, filtered and evaporated under reduced pressure to give a desired 2-deoxy-2,2-difluoro-3,5-bis-(3-fluorobenzoyloxy)-D-ribofuranose (24g, 100%).

1H-NMR (CDCl₃)δ:4.4˜4.75(m, 3H), 5.55(d, 1H), 5.4˜5.7(m, 1H), 7.23˜7.45(m, 4H), 7.70˜7.89(m, 4H)

### Example 4

Preparation of 2-deoxy-2,2-difluoro-D-ribofuranose-3,5-bis-(3-fluorobenzoyloxy)-1-methanesulfonate

To 2-deoxy-2,2-difluoro-3,5-bis-(3-fluorobenzoyloxy)-D-ribofuranose (24 g, 0.057 mole) were added methylene chloride (240 mL) and triethylamine (9.8 g, 0.097 mole) and cooled to 5° C. Methanesulfonyl chloride (7.8 g, 0.068 mole) was mixed to the mixture and stirred for 2 hours. With completion of the reaction, the reaction mixture was washed with a weak solution of hydrochloric acid and water. The mixture was dried over anhydrous sodium sulfate, filtered and evaporated under reduced pressure to give a desired

2-deoxy-2,2-difluoro-D-ribofuranose-3,5-bis-(3-fluorobenzoyloxy)-1-methanesulfonate (28.5 g, 100%).

1H-NMR (CDCl₃)δ:3.10 (s, 3H), 4.67˜4.72 (m, 2H), 4.8 (m, 1H), 5.5 (dd, 1H), 6.1 (d, 1H), 7.24˜7.46 (m, 4H), 7.70˜7.85 (m, 4H)

### Example 5

Preparation of 2′,2′-difluoro-3′,5′-bis-(3-fluorobenzoyloxy)-2′-deoxycytidine

To cytosine (63.2 g, 0.57 mole) was added 1,1,1,3,3,3-hexamethyldisilazane (316 mL), ammonium sulfate (7.5 g, 0.057 mole). The mixture was stirred under reflux for 2 hours and with the addition of 2-deoxy-2,2-difluoro-D-riboftiranose-3,5-bis-(3-fluorobenzoyloxy)-1-methansulfonate (28 g, 0.057 mole), was further stirred under reflux. With completion of the reaction, isopropyl alcohol (63.2 mL) and a weak solution of bromic acid were added to the reacting mixture and stirred at 60° C. for about 1 hour. The mixture was cooled, centrifuged and washed with water and isopropyl alcohol. The crystals, so formed, was dried over a heat wind and dissolved in methanol (160 mL). With the addition of 30% ammonia water (2.7 mL), the mixture was evaporated under reduced pressure. Ethyl acetate (500 mL) was added to the concentrate for suspension and washed with water. The organic layer was evaporated under reduced pressure, followed by recrystallization with ethyl acetate to give, in greater than 99% purity, a beta-anomer 2′,2′-difluoro-3′,5′-bis-(3-fluorobenzoyloxy)-2′-deoxycytidine (10.4 g, 36%).

1H-NMR (CDCl₃)δ:4.53 (m, 1H), 4.71˜4.75 (m, 2H), 5.60 (m, 1H), 5.71 (d, 1H), 6.60 (m, 1H), 7.24˜7.87 (m, 8H)

### Example 6

Preparation of 2′-deoxy-2′,2′-difluorocytidine

To 2′,2′-difluoro-3′,5′-bis-(3-fluorobenzoyloxy)-2′-deoxycytidine (10.4 g, 0.02 mole) were added methanol (104 mL) and 30% ammonia water (20.8 mL). The mixture was stirred at room temperature for 3 hours. With completion of the reaction, the reacting mixture was evaporated under reduced pressure. The concentrate was diluted with water (104 mL) and washed with ethyl acetate (100 mL) two times. The aqueous layer was evaporated under reduced pressure to give 2′-deoxy-2′,2′-difluorocytidine (5.4 g, 100%).

1H-NMR (DMSO-d₆)δ:3.60˜3.64 (dd, J=3.6 Hz, 1H), 3.75˜3.78 (dd, 1H), 3.88 (m, 1H), 4.16 (m, 1H), 6.04 (m, H), 6.24 (d, 1H), 8.14 (d, 1H), 8.89 (s, 1H), 10.04 (s, 1H)

### Example 7

Preparation of 2′-deoxy-2′,2′-difluorocytidine Hydrochloride

To 2′-deoxy-2′,2′-difluorocytidine (5.4 g, 0.02 mole) was added ethanol (54 mL) and a strong hydrochloric acid (1.82 mL). The mixture was stirred under reflux for 30 minutes. The reacting solution was cooled, followed by filtration of crystals, so formed. The filtrated crystals was washed with ethanol and dried by a heat wind for 12 hours to give, in greater than 99.9% purity, 2′-deoxy-2′,2′-difluorocytidine hydrochloride(5.5 g, 90%).

1H-NMR (DMSO-d₆)δ:3.60˜3.64 (dd, J=3.6 Hz, 1H), 3.75˜3.78 (dd, 1H), 3.88 (m, 1H), 4.16 (m, 1H), 6.04 (m, 1H), 6.24(d, 1H), 8.14 (d, 1H), 8.89 (s, 1H), 10.04 (s, 1H)

1. A process for an enantiomer mixture of erythro and threo lactones expressed by the following formula 5, wherein 3-R- and 3-S-enantiomer mixture and its protected derivative of alkyl
2,2-difluoro-3-hydroxy-3-(2,2-dialkyldioxolan-4-yl)propionate expressed by the following formula 4 are hydrolyzed in the presence of hydrolysis reagents selected from acetic acid or chloroacetic acid, water and a mixture of organic solvents selected from the group comprising acetonitrile, dioxane, tetrahydrofuran or toluene: 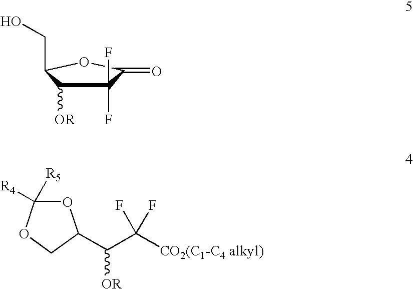
Wherein, R is 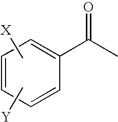
or H; X is F, Cl, Br, I, and NO₂, respectively; Y is H, F, Cl, Br, I and NO₂, respectively, R₄ and R₅ are independently C₁—C₃ alkyl.

2. The process of claim 1, wherein acetic acid or chloroacetic acid, water and a mixture of organic solvents as hydrolysis reagents are mixed in the weight ratio of 10˜95:5˜90:0˜70.

3. A process for selectively isolating, in greater than about 98% purity,
2-deoxy-2,2-difluoro-3,5-bis-(substituted benzoyloxy)-D-erythro-pentofuranos-1-ulose of the following formula 6 from an enantiomeric mixture of erythro and threo lactones of the following formula 6′, comprising the enantiomeric mixture of erythro and threo lactones of the following formula 6′ in ethyl acetate, adding hexane, cooling the solution to a temperature in the range of about 0° C. to −5° C., and collecting the precipitated erythro enantiomer, 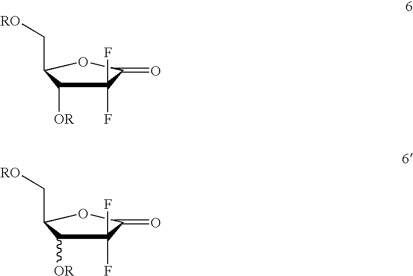
Wherein, R is 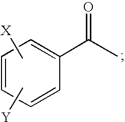
X is F, Cl, Br, I, and NO₂, respectively; and Y is H, F, Cl, Br, I and NO₂, respectively. Further, L is methanesulfonyl and p-toluenesulfonyl.

4. The process of claim 3, comprising the additional step of adding hexane or heptane to the solution of the enantiomeric mixture dissolved in ethyl acetate to provide a hexane/ethyl acetate or heptane/ethyl acetate solvent mixture.

5. A process for purifying, in greater than 98% purity, a beta-anomer 2′-deoxy-2′,2′-difluorocytidine-3′,5′-D-(substituted)-benzoate of the following formula 9 from a compound of the following formula 9′ of alpha- and beta-anomer mixture via recrystallization, comprising reacting a base with silylation reagents to form an enolized compound in the first phase, reacting by heat the protected carbohydrate of the following formula 8 with the enolized compound in the presence of silylation reagents or in the absence of solvent after removing the silylation reagents, and obtaining the compound of the formula 9′, 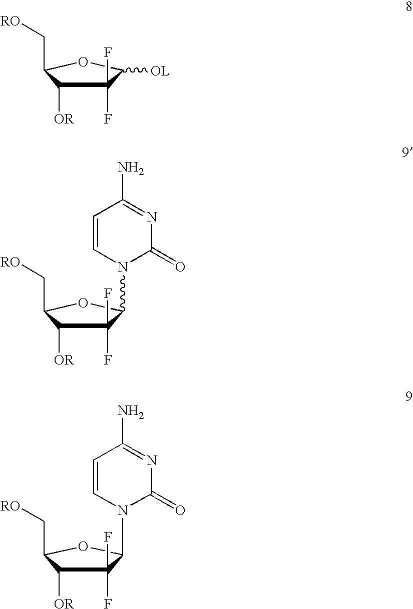
Wherein, R is 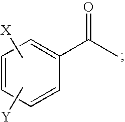
X is F, Cl, Br, I, and NO₂, respectively; and Y is H, F, Cl, Br, I and NO₂, respectively. Further, L is methanesulfonyl and p-toluenesulfonyl.

6. The process of claim 5, wherein the reaction is carried out using a solvent such as hexamethyldisilazane or bistrimethylsilylacetamide.

7. The process of claim 5, wherein the reaction temperature is in the range of 60˜160° C.

8. The process of claim 5, wherein the recrystallization process is carried out using recrystallization solvents such as methanol, ethanol, 2-propanol, ethyl acetate, chlorform and methylene chloride.

9. An erythro compound in greater than 98% purity of the following formula 6, which is isolated by the process of claim 1, 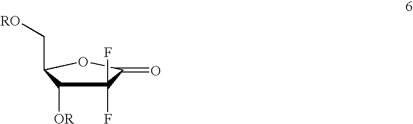
Wherein, R is 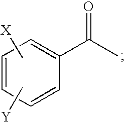
X is F, Cl, Br, I, and NO₂, respectively; and Y is H, F, Cl, Br, I and NO₂, respectively.

10. A Beta-anomer 2′-deoxy-2′,2′-difluorocytidine-3′,5′-D-(substituted)-benzoate of the following formula 9 in greater than 98% purity, which is isolated by the process of claim 5, 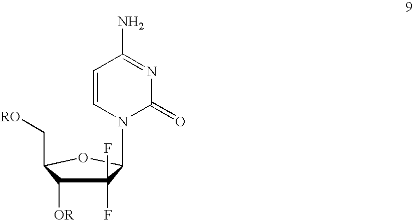
Wherein, R is 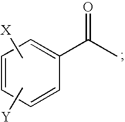
X is F, Cl, Br, I, and NO₂, respectively; and Y is H, F, Cl, Br, I and NO₂, respectively.
